(19) United States
(12) Patent Application Publication
(10) Pub. No.: US 2009/0096987 A1
(43) Pub. Date: Apr. 16, 2009

(54) Eye Measurement Apparatus and a Method of Using Same

(76) Inventors: Ming Lai, Webster, NY (US); Barry T. Eagan, Spencerport, NY (US)

Correspondence Address: Bausch & Lomb Incorporated, One Bausch & Lomb Place, Rochester, NY, 14604-2701, US

(21) Appl. No.: 12/243,361

(22) Filed: Oct. 1, 2008

Related U.S. Application Data
(60) Provisional application No. 60/978,923, filed on Oct. 10, 2007.

(51) Int. Cl. A61B 3/14; A61B 3/113; A61B 3/107

(52) U.S. Cl. 351/206; 351/209

(57) ABSTRACT

An apparatus for measuring a subject's eye having an instrument axis, comprising an eye tracker apparatus comprising a first projector and a first camera, a slit projector rotatable about the instrument axis independent of the eye tracker apparatus, and a second camera rotatable about the instrument axis independent of the eye tracker.

### CROSS-REFERENCE

This application claims the benefit of U.S. Provisional Patent Application No. 60/978,923 filed Oct. 10, 2007, which is incorporated by reference herein.

### FIELD OF INVENTION

The present invention relates to eye measurement apparatus and a method of using same, and more particularly to an eye measurement apparatus including an eye tracker and method of using same.

### BACKGROUND OF THE INVENTION

Ophthalmologists and optometrists would like to have accurate representations of portions of subjects' eyes. Such representations include, for example, representations of a subject's corneal surfaces, corneal thickness, corneal density and lens surfaces. This information may be used, for example, to prescribe contact lenses and eye glasses, and to reshape the cornea by surgical procedures or to perform other surgical procedures. Since it is not comfortable to measure these data by physical contacting an eye, remote sensing techniques are preferably used to obtain the representations.

One common technique for obtaining representations of eyes includes projecting narrow bands of light (commonly referred to as slits or slit beams) onto a subject's cornea at multiple locations on the cornea. For each of the slits, after the light in the slit has been scattered by the eye, an image of the scattered light is obtained. Images from tens of slit projections (e.g., approximately 40 slits of light at different locations) are used to construct representations of one or more portions of the subject's eye.

FIGS. 1 and 2 illustrate one type of measurement apparatus 100 in which slits of light S, S′, at various angular deviations (a) about an instrument axis 102, are projected such that the slits impinge on multiple locations on the cornea C. FIG. 2 is a view of apparatus 100 taken along line 2-2 of FIG. 1. Light scattered by the eye from each slit permits a cross section of the eye to be obtained; and multiple cross sections from slits of different angular deviations permit two-dimensional or three-dimensional representations of the eye to be constructed.

To produce slits of light S, S′, a long, thin aperture 110 (having a length extending in the Y direction in FIG. 1) is placed in front of a source 120 and a beam splitter 125 reflects the light onto the cornea C and lens L along an instrument axis 102. To achieve slits of light S and S′ at the various angular deviations, apparatus 100 (including all components therein) and a portion 170a of the front faceplate 170 of the apparatus is rotated about instrument axis 102. After the light is scattered by the eye, the scattered light re-enters the apparatus through a camera port 135 and is gathered by lens 130 and projected onto a CCD 140 sensor. One image is obtained for each of a plurality of rotational positions of the apparatus.

To help make the measurements more consistent from subject-to-subject, prior to obtaining images, a subject is aligned in front of apparatus 100. An alignment apparatus including two alignment LEDs 152, 154 is arranged to project light onto the cornea. Specularly reflected light from the LEDs passes through beam splitter 125 and is imaged by lens 156 and CCD 158. When the specularly reflected light is in a predetermined position on CCD 158, the subject is assumed to be aligned in the X and Y directions. Images with a slit S extending in the Y direction are obtained using CCD 140 to align the machine in the Z direction. When an image of the slit is in a predetermined position on CCD 140, the subject is assumed to be aligned in the Z direction.

A drawback of such apparatus is that, while a subject is aligned with the machine before beginning the acquisition of images, a subject may move during acquisition of images for constructing a representation. Furthermore, because the slit projector (comprising source 120 and an aperture 110), the slit camera (comprising lens 130 and CCD 140) and LEDs 152, and 154 are rotated to various positions to obtain slits at various angular deviations, it would be difficult or not possible to track a subject's eye during image acquisition (e.g., to determine location of the eye for each image).

In fact, even if eye tracking measurements were attempted after acquisition of the images was begun (i.e., using light from LEDs 152, 154) or light from a slit, since the slit projector, slit camera and the LEDs have been moved (i.e., rotated), it would be difficult to determine if any shift in the alignment images was due to movement resulting from imprecise rotation of the apparatus (e.g., wobble) or due to true misalignment of the patient with the apparatus. Additional uncertainties would arise because patients' eyes are typically not rotationally symmetric; accordingly, a false indication of misalignment may occur due to projection of the beams on different portions of the eye.

### SUMMARY

Aspects of the invention are directed to an apparatus for measuring a subject's eye having an instrument axis, comprising an eye tracker apparatus including a first projector and a first camera, a slit projector rotatable about the instrument axis independent of the eye tracker apparatus, and a second camera rotatable about the instrument axis independent of the eye tracker. In some embodiments the eye tracker is adapted to remain stationary during rotation of the slit projector.

In some embodiments, the slit projector comprises a beam splitter configured to project slits of light along an instrument axis. In some embodiments, the beam splitter is a pellicle. In some embodiments, the beam splitter is a cubic beam splitter having a face disposed perpendicular to the instrument axis.

In some embodiments, the slit projector and the second camera are coupled together so that rotation of the slit projector and the second camera occur by the same angular amount. The eye tracker may be a three-dimensional eye tracker.

In some embodiments, the slit projector is configured and arranged to project slits of light from locations that are remote from the instrument axis. In some embodiments, the apparatus further comprises a shaft disposed along the instrument axis and rotatable about the instrument axis, and at least one of the slit projector and the second camera are connected to the shaft.

### BRIEF DESCRIPTION OF THE DRAWINGS

Illustrative, non-limiting embodiments of the present invention will be described by way of example with reference to the accompanying drawings, in which the same reference number is used to designate the same or similar components in different figures, and in which:

FIG. 1 is a schematic view of a prior art eye measurement apparatus illustrating optical layout;

FIG. 2 is a schematic view of the front of the apparatus of FIG. 1 taken along line 2-2 illustrating the arrangement of the projected slits, the alignment LEDs and the slit camera port;

FIG. 3 is a schematic view of an example of an eye measurement apparatus according to aspects of the present invention illustrating optical layout;

FIG. 4 is a schematic view of the front of the apparatus of FIG. 3 taken along line 4-4 illustrating the arrangement of the projected slits, the alignment projectors and the slit camera;

FIGS. 5A and 5B are schematic views of another example of an eye measurement apparatus according to aspects of the present invention illustrating optical layout; and

FIG. 6 is a schematic view of the front of the apparatus of FIGS. 5A and 5B taken along line 6-6 of FIG. 5A illustrating the arrangement of the projected slits, the alignment LEDs and the slit camera.

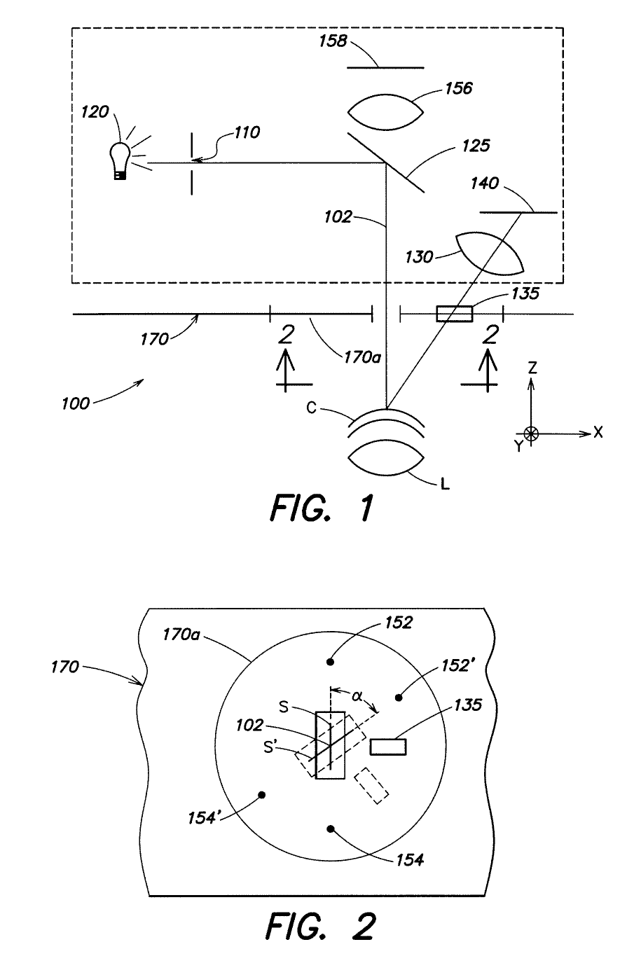

### DETAILED DESCRIPTION

Aspects of the invention are directed to an apparatus for measuring a subject's eye having an instrument axis. The apparatus comprises 1) an eye tracker apparatus comprising a first projector and a first camera, 2) a slit projector rotatable about the instrument axis independent of the eye tracker apparatus, and 3) a second camera for receiving slit light scattered from the eye, the second camera also being rotatable about the instrument axis independent of the eye tracker apparatus. It will be appreciated that, in use, the eye tracker will typically remain stationary during acquisition of images for a given subject to reduce the uncertainty that arises when the eye tracker is rotated; however, the eye tracker may be translatable or rotatable, for example, to calibrate the apparatus.

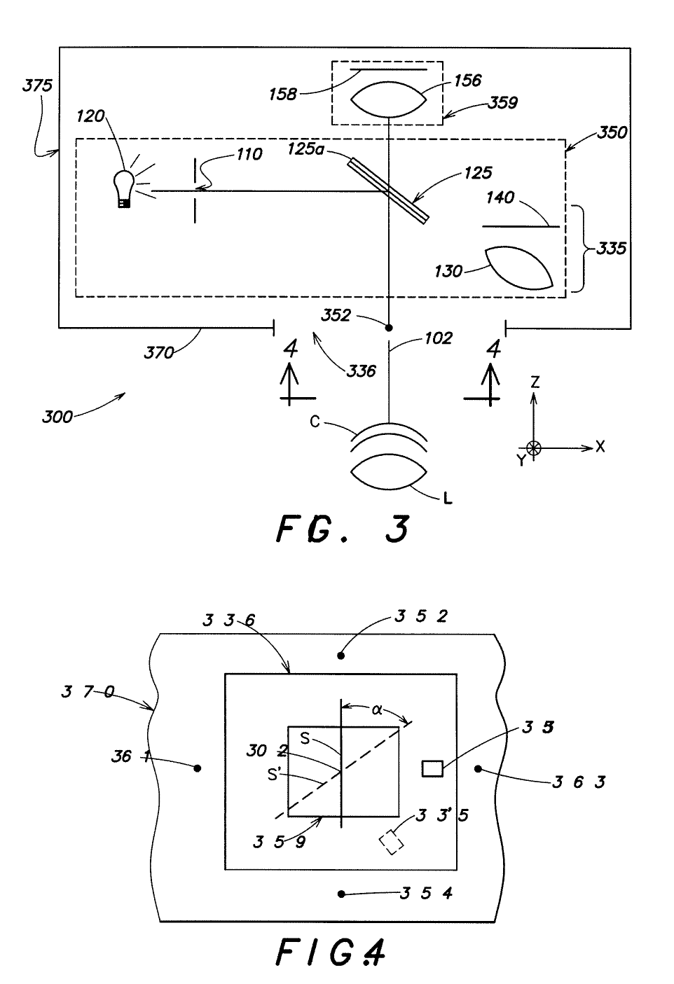

FIG. 3 is a schematic view of an example of an embodiment of an eye measurement apparatus 300 according to aspects of the present invention illustrating optical layout. For example, the eye measurement apparatus is adapted to measure a subject's cornea C and lens L. The measurement apparatus includes an instrument axis 302 about which rotation of slits of light (S, S′ in FIG. 4) occurs. The apparatus comprises a slit projector (comprising source 120, aperture 110 and beam splitter 125), a slit camera 335 (comprising lens 130 and CCD 140), and an eye tracker (comprising a projectors 352 and 354 and camera 359). The slit projector and slit camera together from a slit apparatus 350.

According to aspects of the invention, the rotatable slit projector is rotatable about the instrument axis independent of the eye tracker. The slit projector may be rotatable in any suitable manner that permits multiple cross sections of the eye to be illuminated. For example, the rotation may be in a manner such that the center of the slits is projected along the instrument axis 302 and each of the slits is rotated by an amount about the instrument axis 302. In other embodiments, as discussed in greater detail below, the slits may be projected from a location remote from the instrument axis. Slit projectors, regardless of where they are disposed, are typically configured to project light onto the instruments axis and to rotate such that, at the cornea, each of the slits is a rotational deviation about the instrument axis. Typically, the projector is configured such that slits of light are projected onto the center of a subject's eye, and each of the slits is rotationally deviated from one another.

Slit camera 130, 140 is adapted to receive light after it is scattered from the eye. As shown in FIG. 4, the scattered light passes through a port 336 in a face plate 370. Camera 130, 140 is also rotatable about the instrument axis independent of the eye tracker. Typically, the slit camera is coupled to the slit projector such that rotation of the camera and rotation of the slit projector occur by the same angular amount; however, such an arrangement is not necessary.

In some embodiments, beam splitter 125 is selected to be a pellicle (i.e., a beam splitter having a thin substrate 125a) which will help minimize deviation of the light that is caused by rotating the beam splitter substrate in the path of the light. In some embodiments, beam splitter 125 is selected to be a cubic beam splitter (not shown) having a face disposed perpendicular to the instrument axis to eliminate that deviation of the light that is caused by rotating a beam splitter substrate (having a surface being non-perpendicular to the visual axis) in the path of the light.

The eye tracker comprises a projector system (e.g., projectors 352, 354) and a camera 359 (e.g., lens 156 and CCD 158). Camera 359 is adapted to receive light from projectors 352, 354 after it impinges on the eye. In the illustrated embodiment, camera 359 is adapted to receive light from the LEDs that is specularly reflected from an eye. Accordingly, the eye can be tracked in the X and Y directions in the manner described in the Background above. However, embodiments of the present invention may determine alignment of the apparatus with the eye in any one or more of the X, Y and Z directions for each of the plurality of images to be used to generate a representation of the eye. In some embodiments, it is desirable that alignment is determined in all of the X, Y and Z directions. That is, the eye tracker is a three-dimensional eye tracker.

Beam projectors 361, 363 may be added to determine position in the Z direction. For example, the beam projectors may be arranged to project beams that cross the instrument axis at a predetermined location. Accordingly, the separation of the beams in an image of the cornea obtained by camera 359 will indicate the location of eye relative to predetermined location. The above X-Y and Z tracking devices may be used separate of one another or combined to provide three-dimensional eye tracking. Another example of a suitable three-dimensional eye tracker is given in copending U.S. patent application Ser. No. 11/528,130, by Lai, et al, filed on Sep. 27, 2006 the substance of said application is hereby incorporated by reference in its entirety.

As stated above, the components of slit apparatus 350 are rotatable independent of the eye tracker. In some embodiments, apparatus 300 is configured such that the eye tracker (e.g., including projectors 352, 354 and camera 359) is stationary during collection of images to be used to obtain a representation of the eye. In the illustrated embodiment, the slit apparatus rotates within the apparatus housing 375 and the camera 359 is fixed within the housing.

One or more of the components of the eye tracker may be located on a face plate 370 which remains stationary during image acquisition. For example, in the illustrated embodiment, projectors 354, 354 are so located.

FIG. 4 is a schematic view of the front of the apparatus of FIG. 3 taken along line 4-4 illustrating the arrangement of the projected slits S, S′, alignment projectors 352, 354 and the slit camera 335. Slits S, S′ are shown illustrating rotational deviation (a) of the slits of light caused by rotating slit apparatus 350. Rotational deviation of the camera 335, 335′ corresponding to slit S, S′ is also shown.

It will be appreciated that the arrangement of the apertures in the front of the apparatus and the size of the apertures should be appropriate to permit the light from the eye tracker projector (which may be stationary) and light from slits at each angular deviation (α) to reach the eye. The front of the apparatus should also permit light to reach the eye tracker camera and/or slit camera after the light is scattered by the eye.

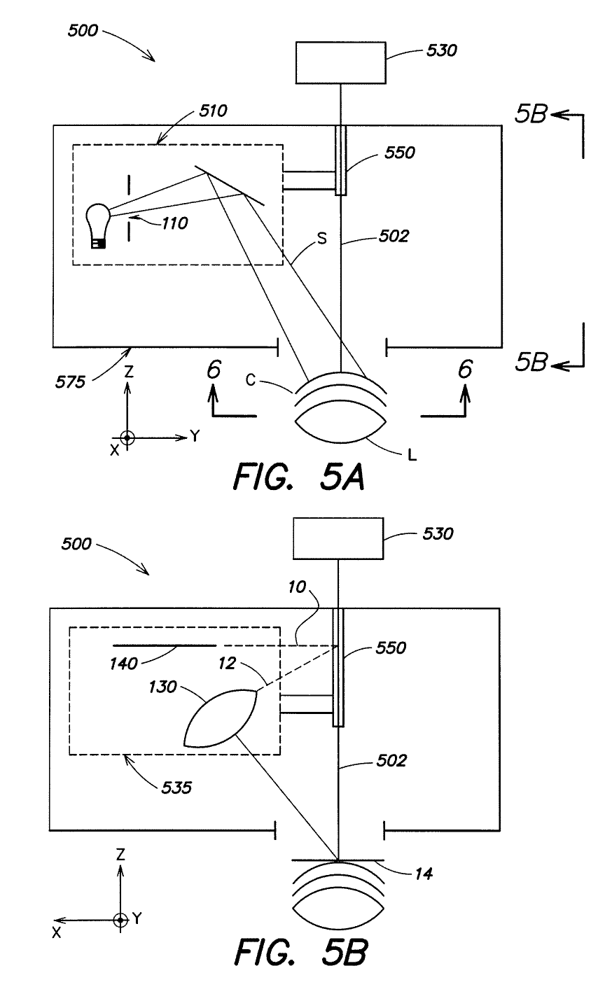

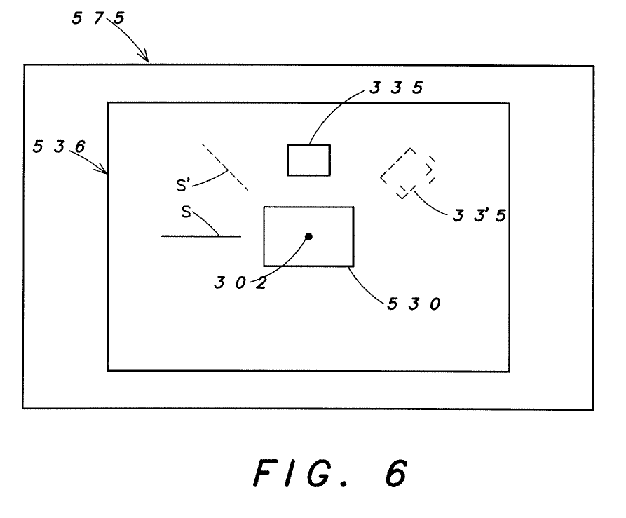

FIGS. 5A, 5B and 6 illustrate another example of an eye measurement apparatus 500 according to aspects of the present invention. FIG. 5B is a view of the apparatus of FIG. 5A taken along lines 5B-5B. FIG. 6 is a view of the instrument face taken along lines 6-6 of FIG. 5A showing the front face of the apparatus and multiple slits S and S′.

Referring to FIG. 5A, apparatus 500 comprises an eye tracker 530 as described above with reference to FIGS. 3 and 4 (comprising a camera and light projector system), and a rotatable slit projector 510 that is rotatable about an instrument axis 502 independent of an eye tracker to provide slits S, S′. Slit projector produces slits of light (shown in FIG. 6) that are projected from locations remote from the instrument axis. The slit projector is configured to project light onto the instruments axis 502 such that each of the slits has a different rotational deviation (a) about the instrument axis. It will also be appreciated that the illustrated embodiment can provide slits of light that impinge on the center of a subject's eye, the slits being rotationally deviated from one another, similar to the apparatus of FIGS. 3 and 4. Provided that common portions of an eye are illuminated, an image of light scattered form the eye from a slit of light projected onto the eye from an off-axis arrangement (as shown in FIGS. 5A, 5B and 6) will be substantially indistinguishable from an image of light scattered by the eye from a slit of light projected onto the eye from an on-axis arrangement (as shown in FIGS. 3 and 4).

Camera 535 (comprising a lens 130 and CCD 140 or other suitable sensor) is also rotatable about the instrument axis independent of the eye tracker. As demonstrated by projection lines 10 and 12, lens 130 and CCD 140 are in a Scheimpflug arrangement so as to have an object plane 14 that is perpendicular to instrument axis 502. In the illustrated embodiment, the eye tracker remains stationary during image acquisition and the slit apparatus (including the slit projector and the slit camera) rotates about the instrument axis 502.

FIG. 6 illustrates the apparatus of FIGS. 5A and 5B taken along line 6-6 of FIG. 5A. FIG. 6 shows slits of light S, S′, alignment LEDs, and a camera. A port 536 is provided in a face plate of housing 575.

It is to be appreciated that an off-axis, slit projection arrangement as shown in FIG. 5A permits omission of the beam splitter 125 (shown in FIGS. 1 and 3). It is to be appreciated that, by avoiding projecting light onto alignment CCD through a rotating slab of glass (e.g., a beam splitter substrate), deviation of the light caused by the rotating slab of glass in the path of the light is eliminated, thereby simplifying tracking and improving accuracy of the tracking apparatus.

It will be also appreciated that an off-axis slit projection arrangement as shown in FIG. 6 makes it possible to include a shaft 550 along the rotational axis of the slit apparatus without obstructing slit projection. The shaft is rotatable about the instrument axis. In some embodiments, it is advantageous to connect the slit projector and/or the slit camera to the shaft to add stability to the rotation (e.g. to reduce wobble of the slit apparatus). Any suitable technique may be used to connect the eye tracker to the shaft. Such techniques will typically provide a rigid connection to avoid wobble. By making the shaft suitably small, no interference with the light from the eye tracker will occur.

It will be still further appreciated that an eye is less sensitive to light projected from an off-axis position. Accordingly, an eye can be illuminated with a brighter light than if on-axis slits are projected; alternatively, a larger pupil size can be attained using the same brightness.

Having thus described the inventive concepts and a number of exemplary embodiments, it will be apparent to those skilled in the art that the invention may be implemented in various ways, and that modifications and improvements will readily occur to such persons. Thus, the embodiments are not intended to be limiting and presented by way of example only. The invention is limited only as required by the following claims and equivalents thereto.

What is claimed is:

1. An apparatus for measuring a subject's eye having an instrument axis, comprising:
an eye tracker comprising a first projector and a first camera;
a slit projector rotatable about the instrument axis independent of the eye tracker; and
a second camera rotatable about the instrument axis independent of the eye tracker.

2. The apparatus of claim 1, wherein the eye tracker is adapted to remain stationary during rotation of the slit projector.

3. The apparatus of claim 1, wherein the slit projector comprises a beam splitter configured to project slits of light along an instrument axis.

4. The apparatus of claim 1, wherein the slit projector and the second camera are coupled together so that rotation of the slit projector and the second camera occur by the same angular amount.

5. The apparatus of claim 1, wherein the eye tracker is a three-dimensional eye tracker.

6. The apparatus of claim 1, wherein the slit projector configured and arranged to project slits of light from locations that are remote from the instrument axis.

7. The apparatus of claim 1, further comprising a shaft disposed along the instrument axis and rotatable about the instrument axis, and at least one of the slit projector and the second camera are connected to the shaft.

8. The apparatus of claim 3, wherein the beam splitter comprises a pellicle.

9. The apparatus of claim 3, wherein the beam splitter comprises a cubic beam splitter having a face disposed perpendicular to the instrument axis.
